(19) United States
(12) Patent Application Publication
(10) Pub. No.: US 2009/0098209 A1
(43) Pub. Date: Apr. 16, 2009

(54) PHARMACEUTICAL COMPOSITIONS CONTAINING WATER-SOLUBLE DERIVATIVES OF PROPOFOL AND METHODS OF ADMINISTERING SAME VIA PULMONARY ADMINISTRATION

(75) Inventors: Roger A. RAJEWSKI, Lawrence, KS (US); Michelle P. McIntosh, Warrandyte (AU)

Correspondence Address: BIRCH STEWART KOLASCH & BIRCH, PO BOX 747, FALLS CHURCH, VA, 22040-0747, US

(73) Assignee: UNIVERSITY OF KANSAS, Lawrence, KS (US)

(21) Appl. No.: 12/251,359

(22) Filed: Oct. 14, 2008

Related U.S. Application Data
(60) Provisional application No. 60/980,150, filed on Oct. 15, 2007.

(51) Int. Cl. A61K 9/14; A61K 31/661; A61K 9/12

(52) U.S. Cl. 424/489; 514/130; 424/45; 424/46

(57) ABSTRACT

The present invention is directed to methods of delivering propofol derivative compounds via pulmonary administration to a mammal in order to induce or maintain anesthetized, sedated and sub-hypnotic states.

This application claims the benefit of U.S. Provisional Application No. 60/989,150, filed Oct. 15, 2007, the entire contents of which are hereby incorporated by reference.

### FIELD OF THE INVENTION

The present invention is directed to methods of delivering propofol derivatives to a mammal via pulmonary administration.

### BACKGROUND OF THE INVENTION

Propofol (2,6-diisopropylphenol) is a low molecular weight phenol derivative that is widely used as an anesthetic, sedative or sub-hypnotic agent by intravenous administration in humans and animals. Among its useful characteristics as an anesthetic drug are: rapid onset and offset of anesthesia, rapid clearance and a side-effect profile that makes it preferable to other anesthetics, such as barbiturates.

In addition to its anesthetic effects, propofol has a range of other biological and medical applications. For example, it has been reported to be an anti-emetic (McCollum J S C et al., Anesthesia 43 (1988) 2391), an anti-epileptic (Chilvers C R, Laurie P S, Anesthesia 45 (1990) 9951) and an anti-pruritic (Borgeat et al., Anesthesiology 76 (1992) 5101). These applications of propofol are typically observed at sub-hypnotic doses. (Borgeat et al., Anesthesiology 80 (1994) 6421). It has also been hypothesized that propofol, due to its antioxidant properties, may be useful in the treatment of inflammatory conditions, especially inflammatory conditions with a respiratory component, and in the treatment of neuronal damage related to neurodegeneration associated with the generation of reactive oxygen species (see, e.g. U.S. Pat. No. 6,254,853 to Hendler et al.).

Propofol, however, is poorly water-soluble, and injection or intravenous delivery of oil-in-water propofol emulsions is associated with undesirable hypertriglyceridemia and frequently causes pain at the injection site, which must be alleviated by the use of a local anesthetic (Dolin S J, Drugs and pharmacology. In: N. Padfield, Ed., Total Intravenous Anesthesia. Butterworth Heinemann, Oxford 20001, and Fulton B and Sorkin E M, Drugs 50 (1995) 6361). Propofol injection is also known to have a hypertensive effect associated with an injection bolus that can require the use of controlled infusion.

Others have attempted to overcome the traditional problems associated with injection or intravenous delivery of oil-in-water propofol emulsions by formulating compositions that enable propofol delivery orally, via inhalation or by creating easily injectable, water-soluble propofol derivative compounds. For example, U.S. Pat. No. 5,496,537 describes solubilizing pure propofol oil in hydrofluorocarbon propellants without the use of surfactants and co-solvents. U.S. Pat. No. 5,288,597 relates, for example, to the delivery of propofol in the form of a medicament composition capable of being absorbed through the mucosal tissues of the mouth, pharynx and esophagus. This patent also describes the incorporation of propofol into a flavored dissolved matrix and the delivery of the drug to the patient through the mucosal tissues.

U.S. Pat. No. 6,204,257 (hereinafter the “'257 patent”) to Stella et al. describes, for example, water soluble and stable derivatives of propofol. The propofol derivatives described in the '257 patent, the contents of which are hereby incorporated by reference, are described to be administered by intravenous injection. The '257 patent describes the preparation of novel, water-soluble phosphonooxymethyl derivatives of alcohol and phenol containing pharmaceuticals represented by the general Formula I:

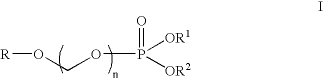

### General Formula I of Phosphonooxymethyl Derivatives of Alcohol and Phenol Containing Pharmaceutical Compounds

In the above Formula I, n represents an integer of 1 or 2; R represents the residue of an alcohol or phenol containing drug, such as propofol; R¹ is hydrogen or an alkali metal ion including sodium, potassium or lithium or a protonated amine or protonated amino acid or any other pharmaceutically acceptable cation; and R² is hydrogen or an alkali metal ion including sodium, potassium or lithium or a protonated amine or protonated amino acid or any other pharmaceutically acceptable cation.

One group of derivatives described in the '257 patent are the propofol derivatives of the Formula II:

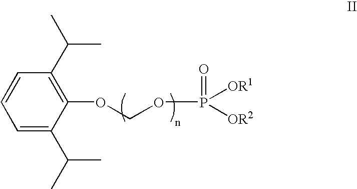

wherein n represents an integer of 1 or 2; R¹ is hydrogen or an alkali metal ion including sodium, potassium or lithium or a protonated amine or protonated amino acid or any other pharmaceutically acceptable cation; and R² is hydrogen or an alkali metal ion including sodium, potassium or lithium or a protonated amine or protonated amino acid or any other pharmaceutically acceptable cation.

One of the propofol derivatives identified in this patent is O-phosphonooxymethyl propofol. This compound is also named phosphoric acid mono-(2,6-diisopropyl-phenoxymethyl)ester. The '257 patent also discloses the disodium salt of O-phosphonooxymethyl propofol.

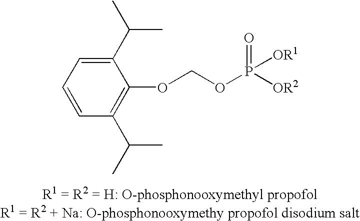

There has been some prior success in the pulmonary administration of low molecular weight drugs, most notably in the area of beta-androgenic antagonists to treat asthma. Other low molecular weight compounds, including corticosteroids and cromolyn sodium, have been administered systemically via pulmonary administration as well. However, many low molecular weight drugs cannot be administered through the lung, and it is not at all predictable that delivery of low molecular weight compounds via pulmonary administration will be effective. For instance, pulmonary administration of aminoglycoside antibiotics, anti-viral drugs, anticancer drugs and many other small molecules for systemic action has proven unsuccessful for a variety of reasons.

Various factors intrinsic to drug compounds, the delivery device and the lung (or a combination of these factors) influence the success of pulmonary administration. The lung presents several barriers to pulmonary administration. Inhaled air (and any particles contained therein) moves into the respiratory tree, which is composed of numerous dichotomous branches between the trachea and the alveoli. The more distal levels of branching form the transitional and respiratory zones, which are comprised of respiratory bronchioles, alveolar ducts and alveoli and the site of gas exchange and pulmonary absorption. The air-blood barrier, central to pulmonary absorption, is comprised of the alveolar epithelium, the capillary endothelium, and the lymph-filled interstitial space separating these two cell layers. (Gehr et al. (1978), Resp. Physiol., Vol. 32, pp. 121-1401). In the alveolar epithelium, adjacent cells overlap and are bound by non-leaky tight junctions, which, in conjunction with the non-leaky single cell layer comprising the capillary endothelium, blocks the movement of fluids, cells, salts, proteins, and numerous other small molecules and macromolecules from the blood and intercellular spaces into the lumen of the alveoli and vice versa. In the absence of lung injury, most molecules must be actively or passively transported across this barrier.

In addition, certain lung epithelial cells secrete mucous to form a contiguous aqueous lining throughout the lung. This layer of moisture, with its incumbent surface tension within the alveoli, generally requires that a surfactant be secreted to reduce surface tension and prevent collapse of the alveoli. In mammals, this surfactant, comprised mostly of lipid, appears to be made up of five layers (Stratton, C. J., Cell Tissue Res., vol. 193, pp. 219-229 (1978)) and can block small molecule drug transport across the air-blood barrier.

Another barrier to the efficacy of pulmonary administration of small molecule drugs can be the ciliary rejection current established in the conducting zone of the lung. Here, numerous ciliated epithelial cells beat in a rhythmic one-way motion to propel the mucous lining overlaying the conducting airways towards the esophagus, where it is expelled from the respiratory system and moved into the digestive tract. Thus, particles impacting on these surfaces can be effectively removed prior to their penetration further into the lung.

In view of the various factors above, whether any particular low molecular weight compound would successfully act as a drug having a systemic effect when delivered via pulmonary administration cannot be predicted a priori.

### BRIEF SUMMARY OF THE INVENTION

The present invention provides methods for the pulmonary administration to a mammal, such as a human, of a therapeutically effective amount of a compounds of Formula II, or a pharmaceutically acceptable salt thereof, which results in the systemic delivery of propofol. Pulmonary administration of a compound according to Formula II provides an effective, non-invasive alternative to the systemic delivery of propofol by injection. The ability to deliver the compounds of Formula II via inhalation enables the treatment of patients in non-clinical settings and in other situations where intravenous administration is difficult to achieve. Pulmonary administration also bypasses the “first pass” metabolism often observed with oral delivery.

The instant invention is based upon the unexpected discovery that compounds of Formula II may be delivered in therapeutically effective amounts by direct administration to the lungs of a patient (hereinafter “pulmonary administration”). Compounds of Formula II or pharmaceutically acceptable salts thereof delivered by pulmonary administration allow for the absorption of propofol into the patient's bloodstream and its systemic distribution. While the present invention is not bound by any theory, the compounds according to Formula II are believed to undergo hydrolysis by cell surface alkaline phosphatases in the lung, resulting in the release of propofol. (Revill, P, Serradell, N, Bolos, J, Drugs Fut. 31 (2006) 859.) The propofol is then rapidly delivered systemically in therapeutically effective amounts without need for injection. Pulmonary administration of a compound according to Formula II provides as another benefit a more convenient self-administration by a patient than does delivery by injection.

The present invention provides methods for inducing, or inducing and maintaining, in a patient an anesthetized or sedated state. In some embodiments, the invention provides methods for the treatment of pain, nausea and vomiting, and epilepsy. Other embodiments of the present invention provide methods for the pulmonary administration of the compounds according to Formula II, or pharmaceutically acceptable salts thereof, in combination with the delivery of one or more additional therapeutic agents. Such additional therapeutic agents may be delivered via pulmonary, oral, injection or other means of administration. Examples of effects provided by additional therapeutic agents include, but are not limited to, induction or maintenance of anesthetized sedated, or hypnotic states; suppression of nausea and/or vomiting; control of epilepsy; muscle relaxation; anti-oxidant effects; anti-emetic effects; anti-pruritic effects; anti-inflammatory effects; analgesia and amnesic effects.

The compounds according to Formula II of the present invention also have non-medical uses. Delivery of the compounds of Formula II via pulmonary administration rapidly induces sedation, and patients suffer few residual effects from the drug upon recovery. The compounds of the invention, therefore, may be used, by way of example, for self-defense, the control of unruly patients or prisoners in medical or penal institutions, crowd or riot control or counter-terrorism purposes.

### BRIEF DESCRIPTION OF THE DRAWINGS

FIG. 1 shows the mean measured plasma concentrations of propofol derived from pulmonary administration of a set amount of O-phosphonooxymethyl propofol disodium salt in a male beagle dog. This study was conducted to demonstrate the usefulness of inhalation delivery of an aqueous solution of O-phosphonooxymethyl propofol disodium salt in producing clinically relevant plasma propofol levels.

FIG. 2 shows the mean measured plasma concentrations of propofol derived from pulmonary delivery for a defined time of O-phosphonooxymethyl propofol disodium salt in male beagle dogs. This study was conducted to demonstrate the usefulness of inhalation delivery of an aqueous solution of O-phosphonooxymethyl propofol disodium salt in producing clinically relevant plasma propofol levels.

### DETAILED DESCRIPTION OF THE INVENTION

Definitions

Throughout the specification and claims, the following definitions apply.

“Effective amount” refers to the amount required to produce a desired effect. In some embodiments, the desired effect is induction of general anesthesia. In some embodiments the desired effect is maintenance of general anesthesia. In some embodiments, the desired effect is induction of a sedated state. In some embodiments the desired effect is maintenance of a sedated state.

“Pharmaceutically acceptable” refers to those properties and/or substances that are acceptable to the patient or medical caregiver from a pharmacological and/or toxicological point of view, and/or to the manufacturing pharmaceutical chemist from a physical and/or chemical point of view regarding composition, formulation, stability, patient acceptance, bioavailability and compatibility with other ingredients.

“Pharmaceutically acceptable salt” refers to an acid or base salt of a compound of the invention, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Pulmonary administration” refers to delivery or administration of an agent to the pulmonary cavity.

“Subject” refers to an animal, including a human. A subject may also be referred to as a “patient.”

“Animal” refers to a living organism having sensation and the power of voluntary movement, and which requires for its existence oxygen and organic food.

“Mammal” refers to a warm-blooded vertebrate animal with hair or fur. Examples include without limitation members of the human, equine, porcine, bovine, murine, canine or feline species.

“General anesthesia” refers to a drug-induced absence of perception of all sensations.

“Sedated state” refers to a state in which the central nervous system is depressed, resulting in calmness, relaxation, sleepiness, slowed breathing, and/or reduction of anxiety.

“Conscious sedated state” refers to a sedated state in which the subject remains conscious.

### Compounds

The present invention provides methods for the pulmonary administration to a mammal, including a human, of an effective amount of the compounds of Formula II, or pharmaceutically acceptable salts thereof:

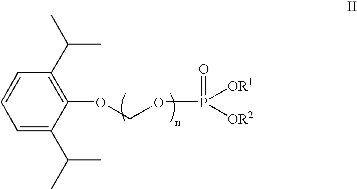

wherein n represents an integer of 1 or 2; R¹ and R² are each independently a member elected from the group consisting of hydrogen, an alkali metal ion (including sodium, potassium or lithium), a protonated amine, and a protonated amino acid.

Useful protonated amines include trimethyamine, triethylamine, triethanolamine, and ethanolamine. Useful protonated amino acids include lysine, arginine, and N-methylglucamine.

In some embodiments, the invention provides methods for the pulmonary administration to a mammal, including a human, of an effective amount of the disodium salt of O-phosphonooxymethyl propofol, or a pharmaceutically acceptable salt thereof:

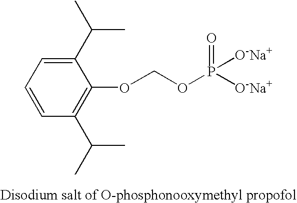

The above compounds can be prepared by techniques described in U.S. Pat. No. 6,204,257 and in U.S. Pat. No. 7,229,978, the contents of which are hereby incorporated in their entirety by reference, and by methods known to those skilled in the art.

### Delivery Methods and Formulations

According to the invention, the propofol compounds of Formula II, or pharmaceutically acceptable salts thereof, are administered via pulmonary administration, that is administration or delivery to the pulmonary cavity. Contemplated modes of pulmonary administration of the compounds according to Formula II include single inhalation administration and continuous inhalation over variable periods of time. The length of inhalation time may be tailored to the medical purpose for which the compounds of the invention are administered and on the individual patient's needs.

The compounds of Formula II may be administered individually or in combination with one or more additional active agents, such as, for example, anesthetic, sedative, or hypnotic agents; suppressants of nausea, vomiting and/or epilepsy; muscle relaxants; anti-oxidants; anti-emetics; anti-pruritics; anti-inflammatories; analagesics and amnesics. Additional active agents may be incorporated into a pharmaceutical composition containing the compound of Formula II, or may be administered in a separately.

Those skilled in the art will recognize the operating conditions for delivery of a suitable dose of a compound according to Formula II for pulmonary administration via inhalation will vary according to delivery vehicle. For some aerosol delivery systems, such as nebulizers, metered inhalers and powder inhalers, the frequency of administration and operating period will be dictated chiefly by the amount of drug per unit volume in an aerosol comprising compounds of Formula II. In general, higher concentrations of the compounds of the present invention in an aerosol will require shorter operating periods. Some devices, such as metered dose inhalers, may produce higher aerosol concentrations than others and thus will be operated for shorter periods to give the desired result.

The pulmonary administration of the compounds of Formula II or pharmaceutically acceptable salts thereof to a patient may be accomplished in several ways, as will be appreciated by those skilled in the art. For example, the compounds of the invention readily form either wet or dry solution aerosols as well as solid aerosols. Solution aerosols may be created by a nebulizer, in particular jet nebulizers and ultrasonic nebulizers, or by other mechanisms. Solid aerosols may be created by a metered dose inhaler, a powder inhaler or other means known in the art. Typically, each formulation is specific to the type of device employed.

Particle size is a consideration in achieving particle deposition in the distal lung regions. Porush et al., reported that to reach the alveoli, small particles should be 0.5 μm to 7 μm in diameter ((1960) Amer. Pharm. Assoc. Sci. Ed., Vol. 49, p. 701) and such particle sizes are useful for the present invention. Later, the preferred particle size for such deposition was reported to be less than 5 μm in diameter (Newman et al., (1983) Thorax, Vol. 38, p. 8811).

Aerosolizing devices may be useful in the methods of the present invention and may involve the use of an appropriate propellant material. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof.

The propofol derivative compounds of Formula II can be formulated in a variety of pharmaceutical compositions alone or in combination with one or more pharmaceutically acceptable carriers, excipients, diluents, adjuvants and/or pharmaceutically active agents. The pharmaceutical compositions of the present invention contain an effective amount of one or more compounds according to Formula II, or pharmaceutically acceptable salts thereof, typically in concentrations from about 0.5 to about 25% (w/v), preferably from about 1 to about 10% (w/v), of a solution formulation. The pharmaceutical compositions of the present invention contain an effective amount of one or more compounds according to Formula II, or pharmaceutically acceptable salts thereof, typically in concentrations from about 0.5 to about 25% (w/w), preferably from about 1 to about 10% (w/w), of a dry formulation. Any pharmaceutically acceptable medium may be used to prepare the pharmaceutical compositions of the present invention, such as sterile water, physiological saline, or a mixture of water and an organic solvent, such as propylene glycol, ethanol and the like.

The pharmaceutical compositions of the present invention may contain an antioxidant to prevent or reduce oxidative degradation of the propofol derivative compounds into poorly water-soluble compounds and/or to act as an additional pharmaceutically active component in compositions of the invention. When present, the concentration of antioxidant typically range from about 0.1 to about 1% (w/v) in a solution formulation, or from about 0.1 to about 1% (w/w) in a dry formulation. A variety of antioxidants may be used, including without limitation monothioglycerol, glutathione, citric acid, ascorbic acid, sodium metabisulfite, and metal chelators, such as EDTA.

The pharmaceutical compositions used in the present invention may contain surfactants. Suitable surfactants include sorbitan trioleate, lecithin, oleic acid, polyoxyethelene and sorbitan fatty acids.

The pharmaceutical compositions used in the present invention preferably have tonicity, i.e., osmolality, essentially the same as that of normal physiological fluids in order to prevent post-administration tissue swelling and for other beneficial reasons well known to persons of skill in the art Acceptable amounts of tonicity modifier typically ranges from about 0.1 to about 1% (w/v). Suitable tonicity modifiers include, without limitation, sodium chloride, glycerin, boric acid, calcium chloride, dextrose, and potassium chloride.

The pH of the pharmaceutical compositions used in the present invention may be maintained to provide long-term stability and other benefits known to persons of ordinary skill in the art. Suitable pH typically ranges from about 7 to about 10, and preferably is at least about 8.5. The solution may be buffered using any buffer effective in the pH range of from about 7 to about 10, including, without limitation, carbonate, phosphate, borate, or glycine. A preferred buffer is tromethamine (2-amino-2-hydroxyethyl-1,3-propanediol), also commonly referred to as TRIS. The concentration of buffer needed for this purpose most often ranges from about 10 to about 25 mM.

In multi-dose container applications, a preservative, such as benzyl alcohol, may be included in the pharmaceutical compositions of the present invention. The pharmaceutical compositions may contain co-solvents such as polyethylene glycol (PEG 200, PEG 400), propylene glycol, and/or ethanol. Concentrations of co-solvents can vary over a wide range, most often from about 0 to about 20%.

The pharmaceutical compositions used in the present invention may include diluents or carriers. Suitable diluents or carriers include, but are not limited to, saccharides and/or sugar alcohols, e.g. monosaccharides, disaccharides and polysaccharides such as glucose, arabinose, dextrose, fructose, ribose, mannose, sucrose, trehalose, lactose, maltose or dextran, sugar alcohols such as mannitol and mixtures of two or more thereof. A preferred diluent or carrier is lactose, particularly in the form of the monohydrate.

The pharmaceutical compositions used in the present invention may contain or be otherwise administered in combination with one or more additional pharmaceutically active agents. Examples of additional active agents include, without limitation, hypnotic, sedative, anesthetic, analgesic, anti-emetic, anti-epileptic, anti-oxidant, anti-inflammatory, amnesic and muscle relaxant agents.

Non-limiting examples of anesthetic, hypnotic, and sedative agents useful in the invention include, without limitation, thiopentone, methohexitone, diazepam, midazolam, ketamine, etomidate, propofol, droperidol, morphine, pethidine, fentanyl, meperidine, alfentanil, sufentanil and remifentanil as well as propofol derivatives according to Formula II. Suitable amounts of such these active components can be ascertained by persons skilled in the art with the aid of no more than routine experimentation.

Exemplary anti-emetic agents useful in the methods of the present invention are well-known to those skilled in the art, and include, without limitation, anticholinergic agents, antihistaminergic agents, butyrophenones, phenothiazines, cannabinoids, benzamides, glucocorticoids, benzodiazepines, and serotonergic antagonists. Specific antiemetic agents include, for example, atropine, hyoscine, diphenhydramine, prochlorperazine, chlorpromazine, haloperidol, droperidol, tetrahydrocannabinol, metoclopramide, trimethobenzamide, dexamethasone, lorazepam, and odansetron as well as propofol derivatives according to Formula II.

Exemplary anti-pruritic agents useful in the methods of the present invention include, without limitation, antihistamines and corticosteroids as well as propofol derivatives according to Formula II.

Exemplary epilepsy control agents useful in the methods of the present invention are well-known to those skilled in the art, and include, without limitation mephobarbital, pentobarbital, clonazepam, clorazepate, diazepam, tiagabine, gabepentin, ethotoin, phenytoin, carbamazepine, valproic acid, topiramate, zonisamide and lamotrigine, as well as propofol derivatives according to Formula II.

Exemplary muscle relaxant agents useful in the methods of the present invention are well-known to those skilled in the art, and include, without limitation, rocuronium bromide, dantrolene sodium, cyclobenaprine hydrochloride, orphenadine citrate and carisoprodol, as well as propofol derivatives according to Formula II.

Exemplary anti-oxidants agents useful in the methods of the present invention are well-known to those skilled in the art, and include, without limitation, monothioglycerol, glutathione, citric acid, ascorbic acid, sodium metabisulfite, and metal chelators, such as EDTA.

Exemplary anti-inflammatory agents useful in the methods of the present invention are well-known to those skilled in the art, and include, without limitation, beclomethasone, beclomethasone proprionate, fluticasone, fluticasone proprionate, triamcinolone, triamcinolone acetate, budesonide and flunisolide, as well as propofol derivatives according to Formula II.

Exemplary analgesic agents useful in the methods of the present invention are well-known to those skilled in the art, and include, without limitation, acetominiophen, tramadol, lidocaine, promethazine, buprenorphine, nalbuphine, propoxyphene, pethidine, hydromorphone, hydrocodone, oxymorphone, oxycodone, indomethacin, celecoxib, rofecoxib and ibuprofen, as well as propofol derivatives according to Formula II.

Exemplary amnesic agents useful in the methods of the present invention are well-known to those skilled in the art, and include, without limitation, lorazepam, scopolamine, midazolam and flunitrazepam, as well as propofol derivatives according to Formula II.

The pharmaceutical compositions used in the present invention may be packaged, for example, in a glass vial, in a pre-filled syringe, or in an ampoule. The dry powder can be in the form of a capsule, usually of a pharmaceutically acceptable natural or synthetic polymer such as gelatin or hydroxypropyl methylcellulose, the capsule containing a unit dose of the propofol derivative compounds of the present invention.

As will be understood by those skilled in the art, a dry powder contained in a capsule may be inhaled by inserting the capsule in a dry powder inhalation device adapted to pierce a capsule containing the dry powder on actuating the device, thereby releasing the dry powder for inhalation by the user—a dry powder capsule inhaler. Such devices are well known in the art and are commercially available. For example, a suitable inhalation device is described in U.S. Pat. No. 3,991,761, which is incorporated herein by reference, particularly as described in the claims of U.S. Pat. No. 3,991,761 and as described with reference to the drawings of U.S. Pat. No. 3,991,761; this device may be modified by coating the capsule-piercing pins with a polymer, as described in WO99/45987. A preferred inhalation device is one adapted to receive a single capsule containing the dry powder, i.e. a single capsule inhaler, for example the commercially available Aerolizer® inhaler.

### Nebulizer Formulations

In some embodiments, Formula II compound formulations suitable for use with a nebulizer, either jet or ultrasonic, comprise the compound of Formula II or a pharmaceutically acceptable salt thereof dissolved in water. In some embodiments the compound of Formula II is dissolved at a concentration of from about 0.1 to about 250 mg of compound per mL of solution. In some embodiments, the compound of Formula II is dissolved in water at a concentration of from about 0.1 to about 50 mg of compound per mL of solution. Such concentration ranges are for illustrative purposes only and not meant to be limiting in any way.

A nebulizer formulation may also include a buffer. Examples of buffers that may be used are TRIS, carbonate, phosphate, borate, or glycine. Preferably, the buffer will have a composition and molarity suitable to adjust the solution to a pH in the range of from about 7 to about 10. Generally, buffer molarities of from about 10 mM to about 25 mM are suitable for this purpose.

A nebulizer formulation may also contain a surfactant. Various conventional surfactants can be employed, such as polyoxyethylene fatty acid esters and alcohols, and polyoxyethylene sorbitan fatty acid esters. In some embodiments the surfactant is present in an amount of between about 0.001% and about 4% (w/v) of the formulation. An especially preferred surfactant for purposes of this invention is polyoxyethylene sorbitan monooleate.

Two nonlimiting examples examples, for illustrative purposes only, of commercially available nebulizers suitable for the practice of this invention are the Ultravent nebulizer, manufactured by Mallinckrodt, Inc., St. Louis, Mo., and the Acorn II nebulizer, manufactured by Marquest Medical Products, Englewood, Colo.

### Metered Dose Inhaler Formulations

In some embodiments formulations for use with a metered dose inhaler device comprise a finely divided dry powder of a compound of Formula II. In some embodiments, sugars or sugar alcohols may be added to the formulation. Nonlimiting examples of such sugars and sugar alcohols include lactose maltose, mannitol, sorbitol, sorbitose, trehalose, xylitol, and xylose. The amount added to the formulation can range from about 0.01 to about 200% of the weight of the compound of Formula II present in the formulation. In some embodiments, the sugars or sugar alcohols may be present in amounts from about 1 to about 50% of the weight of the compound of Formula II present in the formulation. In some embodiments the compound of Formula II is prepared in particulate form with an average particle size of less than about 10 μm (or micrometers). In some embodiments, the compound of Formula II is prepared in particulate form with an average particle size of from about 1 to about 5 μm.

In some embodiments the particles are suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant. This mixture is then loaded into the delivery device. A nonlimiting example, for illustrative purposes only, of a commercially available metered dose inhaler suitable for use in the present invention is the Ventolin metered dose inhaler, manufactured by Glaxo Inc., Research Triangle Park, N.C.

### Powder Inhaler Formulation

In some embodiments formulations for powder inhalers will comprise a finely divided dry powder containing a compound of Formula II, or a pharmaceutically acceptable salt thereof. Some embodiments include a bulking agent, such as lactose, sorbitol, sucrose, or mannitol in amounts which facilitate dispersal of the powder from the device. In some embodiments, the surfactant is present in an amount of between about 50% and about 90% of the weight of the compound of Formula II present in the formulation. In some embodiments the compound of Formula II is prepared in particulate form with an average particle size of less than about 10 μm (or micrometers). In some embodiments, the compound of Formula II is prepared in particulate form with an average particle size of from about 1 to about 5 μm. A nonlimiting example, for illustrative purposes only, of a powder inhaler suitable for use in accordance with the teachings herein is the Spinhaler powder inhaler, manufactured by Fisons Corp., Bedford, Mass.

### Conditions Treated

An anesthetized state achieved by the pulmonary administration of compounds according to Formula II of the present invention is useful for the control in a patient of acute pain induced by surgery or other injury. The unconscious-sedated, conscious-sedated, hypnotic and sub-hypnotic states achieved by pulmonary administration of compounds according to Formula II of the present invention are useful for a variety of medical purposes and treatments. Examples of such uses include, but are not limited to, suppression of nausea and/or vomiting; control of epilepsy; muscle relaxant; anti-oxidant; anti-emetic; anti-pruritic; anti-inflammatory; analgesic and amnesic. Periods of conscious sedation provided by the methods of the present invention are desirable, for example, in procedures that tend to have an unsettling effect on the patient, e.g., imaging studies during which patients are confined to a narrow NMR tube for extended periods; colonoscopy; surgery under spinal or local anesthesia; eye surgery; arthroscopic surgery and the like.

It has been found that plasma propofol derived from pulmonary delivery of the propofol derivative compounds according to Formula II of the present invention appears rapidly and at clinically relevant levels. One embodiment of the present invention provides a method for inducing or maintaining a state of general anesthesia in a patient by pulmonary administration of a compound according to Formula II, or a pharmaceutically acceptable salt thereof, in an amount of from about 10 to about 50 mg per kilogram of body weight.

The induction of general anesthesia through a single dose may require a higher dose than maintenance of anesthesia through subsequent doses. Thus, in some embodiments a single inhalation dose of from about 10 to about 50 mg/kg of body weight of a compound of Formula II or a pharmaceutically acceptable salt thereof may be used to cause loss of consciousness in a patient. In some embodiments a single inhalation dose of from about 15 to about 30 mg/kg body weight of a compound of Formula II or a pharmaceutically acceptable salt thereof may be used to cause loss of consciousness in a patient. Once induced, the anesthetized state may thereafter be maintained by one or more subsequent doses of a compound according to Formula II or a pharmaceutically acceptable salt thereof, administered by pulmonary administration. In some embodiments each subsequent dose is independently of from about 10 to about 20 mg/kg of body weight, via pulmonary administration.

In some embodiments, each single inhalation dose of the compound of Formula II for the purpose of inducing or maintaining general anesthesia may be independently administered for a duration of from about 0.1 sec to about 45 minutes. In some embodiments, each single inhalation dose of the compound of Formula II may be independently administered for a duration of from about 0.1 sec to about 10 minutes. In some embodiments, dosage rates of each dose of the compound of Formula II are independently from about 1 to about 35 mg/min. In some embodiments, dosage rates of each dose of the compound of Formula II are independently from about 10 to about 35 mg/min. In some embodiments, dosage rates of each dose of the compound of Formula II are independently from about 15 to about 30 mg/min. In some embodiments, dosage rates of each dose of the compound of Formula II are independently from about 15 to about 20 mg/min.

In another embodiment of the present invention sedated state is induced or maintained in a patient. In some embodiments the sedated state is a conscious sedated state. The sedated state can be induced or maintained by the pulmonary administration of an effective amount of a compound according to Formula II or a pharmaceutically acceptable salt thereof. The induction of a sedated state via pulmonary administration of compounds according to Formula II may require a higher dose than the maintenance of an existing sedated state. In some embodiments, a sedated state may be induced in a patient by pulmonary administration of a dose of a compound according to Formula II, or a pharmaceutically acceptable salt thereof, of from about 2 to about 20 mg/kg of body weight. In some embodiments, a conscious sedated state may be induced in a patient by pulmonary administration of a dose of a compound according to Formula II, or a pharmaceutically acceptable salt thereof, of from about 2 to about 20 mg/kg of body weight. In some embodiments, a sedated state may be induced in a patient by pulmonary administration of an initial dose of a compound according to Formula II, or a pharmaceutically acceptable salt thereof, of from about 5 to about 10 mg/kg of body weight. In some embodiments, a conscious sedated state may be induced in a patient by pulmonary administration of an initial dose of a compound according to Formula II, or a pharmaceutically acceptable salt thereof, of from about 5 to about 10 mg/kg of body weight. Once induced, the sedated or conscious sedated state may thereafter be maintained by one or more subsequent doses of a compound according to Formula II, or a pharmaceutically acceptable salt thereof, administered by pulmonary administration. In some embodiments each subsequent dose is independently of from about 2 to about 20 mg/kg of body weight via pulmonary administration. In some embodiments each subsequent dose is independently of from about 2 to about 4 mg/kg of body weight via pulmonary administration.

In some embodiments, each single inhalation dose of the compound of Formula II for the purpose of inducing or maintaining sedation or conscious sedation may be administered independently for a duration of from about 0.1 sec to about 45 minutes. In some embodiments, each single inhalation dose of the compound of Formula II may be administered independently for a duration from about 0.1 sec to about 10 minutes. In some embodiments, dosage rates of each dose of the compound of Formula II are independently from about 1 to about 35 mg/min. In some embodiments, dosage rates of each dose of the compound of Formula II are independently from about 10 to about 35 mg/min. In some embodiments, dosage rates of each dose of the compound of Formula II are independently from about 15 to about 30 mg/min. In some embodiments, dosage rates of each dose of the compound of Formula II are independently from about 15 to about 20 mg/min. In some embodiments, dosage rates of each dose of the compound of Formula II are independently from about 5 to about 15 mg/min.

Another aspect of the present invention provides a method for inducing a hypnotic state, i.e. a state of sedation and anesthesia, in a patient by the pulmonary administration of compounds according to Formula II. Appropriate doses for this purpose typically range from about 0.1 to 40 mg/kg, more usually from about 1 to 30 mg/kg, and even more usually from about 5 to 20 mg/kg body weight.

Another aspect of the present invention provides a method of inducing a sub-hypnotic state, i.e. a state that does not provide anesthesia, in a patient by the pulmonary administration of compounds according to Formula II. Appropriate doses for this purpose typically range from about 0.1 to about 15 mg/kg, preferably from about 1 to 10 mg/kg, and more preferably from about 1 mg/kg to 5 mg/kg. Appropriate rates of inhalation typically range from about 1 mg/min to 20 mg/min, more typically from about 2 mg/min to 15 mg/min.

The dosages of the compounds of Formula II and the rates of administration described above are exemplary and are not be construed as limiting the invention. As will be apparent to persons skilled in the art, many factors specific to each individual patient that modify the action of the drug will be taken into account in determining dosage including, but not limited to, the age, sex, diet and other physical conditions of the individual patient. Those skilled in the art will be able to ascertain, without undue experimentation, appropriate treatment protocols for administering the compounds of Formula II.

### Biological Evaluations

Study 1

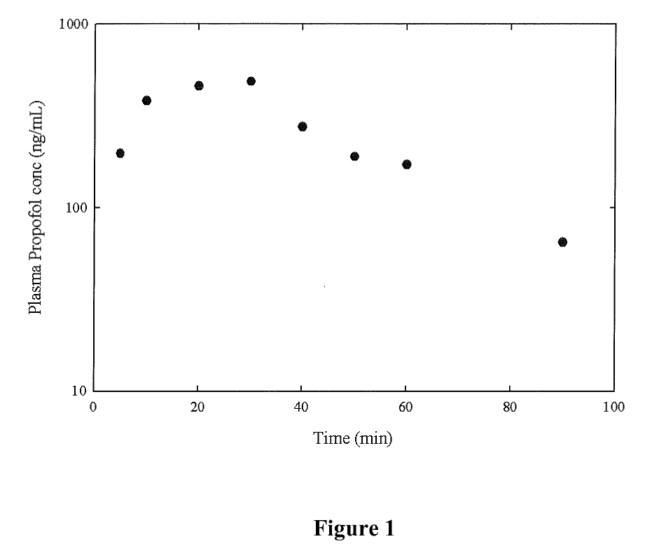

A preliminary study was conducted to assess the conversion of O-phosphonooxymethyl propofol disodium salt to the pharmacologically active drug propofol following pulmonary delivery. A male beagle dog was fasted overnight prior to the study. O-phosphonooxymethyl propofol disodium salt was dissolved in water (250 mg/mL). 3-mL of the O-phosphonooxymethyl propofol disodium salt solution was delivered over approximately 10 min to the dog via inhalation through a nebulizer connected to a facemask that was placed over the dog's muzzle. The actual dose of O-phosphonooxymethyl propofol disodium salt delivered to the dog is less than the dose nebulized due to vapor loss during exhalation. Two mL blood samples were collected via a catheter placed in the cephalic vein prior to the study. Samples were collected for 1.5 hours post-dose at 5, 10, 20, 30, 40, 50, 60 and 90 minutes after initiation of delivery by inhalation. The concentration of propofol in the blood samples was determined using HPLC with fluorescence detection. The results are shown in FIG. 1.

The first blood sample was taken at 5 minutes post dose initiation and there was a significant concentration of propofol in the plasma (see FIG. 1). The dog showed signs of sedation approximately 15 minutes after the completion of the inhaled dose. These finding are significant, as it is known that humans are more susceptible to the affects of propofol than dogs. Therefore, the onset of action would be very rapid in humans.

Study 2

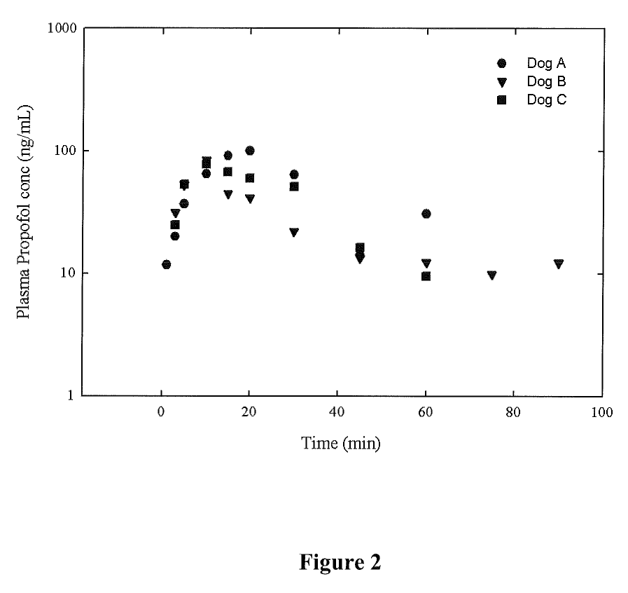

A second study was conducted to assess the conversion of O-phosphonooxymethyl propofol disodium salt to the pharmacologically active drug propofol following pulmonary delivery. The study design was essentially the same as Study 1 above except that the O-phosphonooxymethyl propofol disodium salt solution was nebulized for a defined period of time (10 min) instead of to complete nebulization of the solution. Three male beagle dogs was fasted overnight prior to the study. O-phosphonooxymethyl propofol disodium salt was dissolved in water (250 mg/mL). The O-phosphonooxymethyl propofol disodium salt solution was delivered for 10 min to the dog via inhalation through a nebulizer connected to a facemask that was placed over the dog's muzzle. The actual dose of O-phosphonooxymethyl propofol disodium salt delivered to the dog is less than the dose nebulized due to vapor loss during exhalation. Two mL blood samples were collected via a catheter placed in the cephalic vein prior to the study. Samples were collected for 1.5 hours post-dose at 5, 10, 20, 30, 40, 50, 60 and 90 minutes after initiation of delivery by inhalation. The concentration of propofol in the blood samples was determined using HPLC with fluorescence detection. The results are shown in FIG. 2.

The first blood sample was taken at 5 minutes post dose initiation and there was a significant concentration of propofol in the plasma (see FIG. 2). The dogs showed signs of sedation approximately 10-15 minutes after the completion of the inhaled dose. These finding are significant, as it is known that humans are more susceptible to the effects of propofol than dogs. Therefore, the onset of action would be very rapid in humans.

Although the aerosolized delivery of the propofol derivative in the above-described experiment has been achieved through the use of a nebulizer, it should be understood that other devices such as metered dose inhalers, dry powder inhalers and the like may also be used in the practice of this invention.

### Certain Embodiments of the Invention

1. A method of inducing or maintaining general anesthesia in a mammal, comprising administering to said mammal via pulmonary administration an effective amount of a pharmaceutical composition comprising a compound of Formula II:

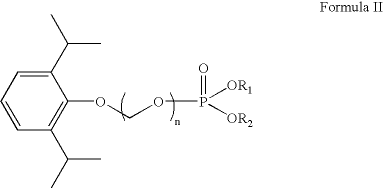

or a pharmaceutically acceptable salt thereof,
wherein n is an integer of 1 or 2; R₁ and R₂ are each independently selected from group consisting of hydrogen, an alkali metal ion, a protonated amine, and a protonated amino acid.
2. The method of embodiment 1, wherein said compound is administered to said mammal in an amount between 10 and 50 mg/kg of the mammal's body weight.
3. The method of embodiment 1, wherein a state of general anesthesia is induced by administration to said mammal of a first dose of said compound and further comprising administration of a second dose of said compound at a dosage level less than said first dose.
4. The method of embodiment 3, wherein said first dose is between 15 and 30 mg/kg of body weight and said second dose is between 10 and 20 mg/kg of the mammal's body weight.
5. The method of embodiment 1, wherein said mammal is a human.
6. A method of inducing or maintaining a sedated state in a mammal, comprising administering to said mammal via pulmonary administration an effective amount of a pharmaceutical composition comprising a compound of Formula II:

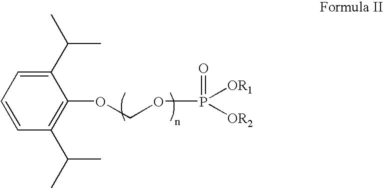

or a pharmaceutically acceptable salt thereof,
wherein n is an integer of 1 or 2; R₁ and R₂ are each independently selected from group consisting of hydrogen, an alkali metal ion, a protonated amine, and a protonated amino acid.
7. The method of embodiment 6, wherein said sedated state is induced by administering to said mammal a dose of said compound that is between 2 and 20 mg/kg of the mammal's body weight.
8. The method of embodiment 6, wherein a sedated state is induced by administering to said mammal a dose of said compound that is between 2 and 20 mg/kg of the mammal's body weight and and further comprising administration of a second dose of said compound at a dosage level of between 2 and 20 mg/kg of the mammal's body weight.
9. The method of embodiment 6, wherein a sedated state is induced by administering to said mammal a dose of said compound that is between 5 and 10 mg/kg of body weight.
10. The method of embodiment 6, wherein said sedated state is induced by administering to said mammal a dose of said compound that is between 5 and 10 mg/kg of the mammal's body weight and and further comprising administration of a second dose of said compound at a dosage level of between 2 and 20 mg/kg of the mammal's body weight.
11. The method of embodiment 6, wherein said mammal is a human.
12. The method of embodiment 1 or embodiment 6, wherein said pharmaceutical composition is an aerosol formulation.
13. The method of embodiment 12, wherein said mammal is a human.
14. The method of embodiment 12, wherein said aerosol formulation further comprises a compound selected from the group consisting of a buffer, a surfactant, a salt, a preservative, a bulking agent, and an antioxidant.
15. The method of embodiment 12, wherein said aerosol formulation is a solution aerosol formulation.
16. The method of embodiment 15, wherein said solution aerosol formulation is administered by a nebulizer, a jet nebulizer or an ultrasonic nebulizer.
17. A pharmaceutical formulation, comprising a compound of Formula II or a pharmaceutically acceptable salt thereof and water, wherein said compound is present in an amount between 0.5% and 25.0% w/v of the formulation.
18. The pharmaceutical formulation of embodiment 17, further comprising a buffer present in sufficient amount to provide a solution pH of from 7 to 10.
19. The pharmaceutical formulation of embodiment 17, further comprising a surfactant present in an amount between 0.001% and 4.000% w/v of the formulation.
20. The pharmaceutical formulation of embodiment 19, wherein the surfactant is polyoxyethylene sorbitan monophosphate.
21. The method of embodiment 12, wherein said aerosol formulation is a powder formulation.
22. The method of embodiment 21, wherein the particles of said powder have diameters of between 0.5 and 7.0 μm.
23. The method of embodiment 21, wherein said aerosol formulation is administered by a metered dose inhaler or a powder inhaler.
24. A pharmaceutical formulation, comprising a suspension of a compound of Formula II or a pharmaceutically acceptable salt thereof in a pharmaceutically acceptable propellant.
25. The pharmaceutical formulation of embodiment 24, wherein the pharmaceutically acceptable propellant is selected from the group consisting of chlorofluorocarbons, hydrochlorofluorocarbons, hydrofluorocarbons, and hydrocarbons; or a combination thereof.
26. The pharmaceutical formulation of embodiment 24, further comprising a surfactant.
27. The pharmaceutical formulation of embodiment 26, wherein the surfactant is selected from the group consisting of sorbitan trioleate, soya lecithin, and oleic acid.
28. A pharmaceutical formulation of embodiment 24, further comprising a sugar or a sugar alcohol.
29. The pharmaceutical formulation of embodiment 28 wherein the sugar or sugar alcohol is, selected from the group consisting of lactose, maltose, mannitol, sorbitol, sorbitose, trehalose, xylitol, and xylose.
30. A pharmaceutical formulation, comprising a compound of Formula II or a pharmaceutically acceptable salt thereof and a bulking agent.
31. The pharmaceutical formulation of embodiment 30, wherein said bulking agent comprises a member selected from the group consisting of lactose, glucose, arabinose, dextrose, fructose, ribose, maltose, trehalose, sucrose, mannose, mannitol, sorbitose, sorbitol, xylose, and xylitol.
32. The pharmaceutical formulation of embodiment 30, wherein said bulking agent is present in an amount of between 50% and 90% w/w of said formulation.
33. The pharmaceutical formulation of embodiments 17, 24 or 30, further comprising an antioxidant.
34. The pharmaceutical formulation of embodiment 33, wherein said antioxidant is present in an amount between 0.1% and 1% w/v of said formulation
35. The pharmaceutical formulation of embodiment 33, wherein said antioxidant comprises at least one member selected from the group consisting of monothiolglycerol, glutathione, citric acid, ascorbic acid, sodium metabisulfite, EDTA and EGTA.
36. The pharmaceutical formulation of embodiment 24 or 30 wherein said compound of Formula II or a pharmaceutically acceptable salt thereof is present in an amount between 0.5% and 50% w/w of the formulation.
37. The method of embodiment 1 or 6, wherein the amine which is protonated is selected from the group consisting of trimethylamine, triethylamine, triethanolamine, and ethanolamine.
38. The method of embodiment 1 or 6, wherein the amino acid which is protonated is selected from the group consisting of lysine, arginine, and N-methylglucamine.
39. The method of embodiment 1 or 6, wherein the alkali metal ion is selected from the group consisting of lithium, sodium, and potassium.
40. The method of embodiment 1 or 6, wherein n is 1.
41. The method of embodiment 1 or 6, wherein the compound of Formula II is O-phosphonooxymethyl propofol disodium salt.

All of the publications and patents cited herein are fully incorporated by reference.

The foregoing examples or preferred embodiments described herein are not meant to limit or restrict the scope of the invention. Persons of skill in the art will recognize that the invention can be practiced with modification within the spirit and scope of the appended claims.

What is claimed is:

1. A method of inducing or maintaining general anesthesia in a mammal, comprising administering to said mammal via pulmonary administration an effective amount of a pharmaceutical composition comprising a compound of Formula II: 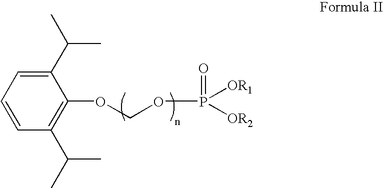
or a pharmaceutically acceptable salt thereof,
wherein n is an integer of 1 or 2; R₁ and R₂ are each independently selected from group consisting of hydrogen, an alkali metal ion, a protonated amine, and a protonated amino acid.

2. The method of claim 1, wherein said compound is administered to said mammal in an amount between 10 and 50 mg/kg of the mammal's body weight.

3. The method of claim 1, wherein a state of general anesthesia is induced by administration to said mammal of a first dose of said compound and further comprising administration of a second dose of said compound at a dosage level less than said first dose.

4. The method of claim 3, wherein said first dose is between 15 and 30 mg/kg of body weight and said second dose is between 10 and 20 mg/kg of the mammal's body weight.

5. A method of inducing or maintaining a sedated state in a mammal, comprising administering to said mammal via pulmonary administration an effective amount of a pharmaceutical composition comprising a compound of Formula II: 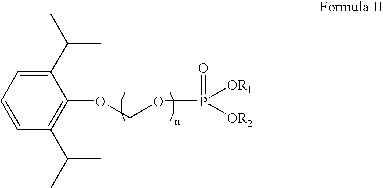
or a pharmaceutically acceptable salt thereof,
wherein n is an integer of 1 or 2; R₁ and R₂ are each independently selected from group consisting of hydrogen, an alkali metal ion, a protonated amine, and a protonated amino acid.

6. The method of claim 5, wherein said sedated state is induced by administering to said mammal a dose of said compound that is between 2 and 20 mg/kg of the mammal's body weight.

7. The method of claim 5, wherein a sedated state is induced by administering to said mammal a dose of said compound that is between 2 and 20 mg/kg of the mammal's body weight and further comprising administration of a second dose of said compound at a dosage level of between 2 and 20 mg/kg of the mammal's body weight.

8. The method of claim 5, wherein a sedated state is induced by administering to said mammal a dose of said compound that is between 5 and 10 mg/kg of body weight.

9. The method of claim 5, wherein said sedated state is induced by administering to said mammal a dose of said compound that is between 5 and 10 mg/kg of the mammal's body weight and further comprising administration of a second dose of said compound at a dosage level of between 2 and 20 mg/kg of the mammal's body weight.

10. The method of claim 1 or claim 5, wherein said pharmaceutical composition is an aerosol formulation.

11. The method of claim 10, wherein said aerosol formulation further comprises a compound selected from the group consisting of a buffer, a surfactant, a salt, a preservative, a bulking agent, and an antioxidant.

12. The method of claim 10, wherein said aerosol formulation is a solution aerosol formulation.

13. The method of claim 12, wherein said solution aerosol formulation is administered by a nebulizer, a jet nebulizer or an ultrasonic nebulizer.

14. A pharmaceutical formulation, comprising a compound of Formula II or a pharmaceutically acceptable salt thereof and water, wherein said compound is present in an amount between 0.5% and 25.0% w/v of the formulation.

15. The pharmaceutical formulation of claim 14, further comprising a buffer present in sufficient amount to provide a solution pH of from 7 to 10.

16. The pharmaceutical formulation of claim 14, further comprising a surfactant present in an amount between 0.001% and 4.000% w/v of the formulation.

17. The pharmaceutical formulation of claim 14, wherein the surfactant is polyoxyethylene sorbitan monophosphate.

18. The method of claim 10, wherein said aerosol formulation is a powder formulation.

19. The method of claim 18, wherein the particles of said powder have diameters of between 0.5 and 7.0 μm.

20. The method of claim 18, wherein said aerosol formulation is administered by a metered dose inhaler or a powder inhaler.

21. A pharmaceutical formulation, comprising a suspension of a compound of Formula II or a pharmaceutically acceptable salt thereof in a pharmaceutically acceptable propellant.

22. The pharmaceutical formulation of claim 21, further comprising a surfactant.

23. A pharmaceutical formulation, comprising a compound of Formula II or a pharmaceutically acceptable salt thereof and a bulking agent.

24. The pharmaceutical formulation of claim 23, wherein said bulking agent comprises a member selected from the group consisting of lactose, glucose, arabinose, dextrose, fructose, ribose, maltose, trehalose, sucrose, mannose, mannitol, sorbitose, sorbitol, xylose, and xylitol.

25. The pharmaceutical formulation of claim 23, wherein said bulking agent is present in an amount of between 50% and 90% w/w of said formulation.

26. The pharmaceutical formulation of any one of claims 14, 21 or 23, further comprising an antioxidant.

27. The pharmaceutical formulation of claim 26, wherein said antioxidant is present in an amount between 0.1% and 1% w/v of said formulation

28. The pharmaceutical formulation of claim 26, wherein said antioxidant comprises at least one member selected from the group consisting of monothiolglycerol, glutathione, citric acid, ascorbic acid, sodium metabisulfite, EDTA and EGTA.

29. The pharmaceutical formulation of claims 21 or 23 wherein said compound of Formula II or a pharmaceutically acceptable salt thereof is present in an amount between 0.5% and 50% w/w of the formulation.

30. The method of claims 1 or 5, wherein the amine which is protonated is selected from the group consisting of trimethylamine, triethylamine, triethanolamine, and ethanolamine.

31. The method of claims 1 or 5, wherein the amino acid which is protonated is selected from the group consisting of lysine, arginine, and N-methylglucamine.

32. The method of claims 1 or 5, wherein the alkali metal ion is selected from the group consisting of lithium, sodium, and potassium.

33. The method of claim 1 or 5, wherein n is 1.

34. The method of claim 1 or 5, wherein the compound of Formula II is O-phosphonooxymethyl propofol disodium salt.
